(19) United States
(12) Patent Application Publication
(10) Pub. No.: US 2019/0224238 A1
(43) Pub. Date: Jul. 25, 2019

(54) TUMOR THERAPEUTIC DRUG

(71) Applicant: HUBEI SOUNDNY BIO TECH CO., LTD., Wuhan, Hubei (CN)

(72) Inventors: Bo HUANG, Wuhan, Hubei (CN); Yi ZHANG, Wuhan, Hubei (CN); Ke TANG, Wuhan, Hubei (CN)

(21) Appl. No.: 16/330,344

(22) PCT Filed: Sep. 4, 2017
(86) PCT No.: PCT/CN2017/100353

§ 371 Date: Mar. 4, 2019

(30) Foreign Application Priority Data
Sep. 5, 2016 (CN) 201610801977.2

(51) Int. Cl. A61K 35/18; A61K 9/16; A61K 31/519; A61P 35/02; A61K 9/10

(52) U.S. Cl. CPC A61K 35/18; A61K 9/1694; A61K 9/10; A61K 9/1664; A61P 35/02; A61K 31/519

(57) ABSTRACT

An erythrocyte vesicle-derived tumor therapeutic drug, including erythrocyte vesicles and a therapeutic drug encapsulated in the erythrocyte vesicles is provided. The erythrocyte vesicles are vesicles released by apoptotic erythrocytes, and the therapeutic drug is a tumor therapeutic drug as an effective component for treating tumors.

### FIELD OF THE INVENTION

The invention relates to a tumor therapeutic drug, and in particular to a tumor chemotherapeutic drug derived from erythrocyte vesicles (i.e. microparticles).

### BACKGROUND OF THE INVENTION

With the deteriorating ecological environment in which human beings live, high incidence of human tumor diseases is induced, and tumor has become a major disease that seriously threatens human health. How to effectively treat it has always been the subject of scientific research. The most conventional method of tumor treatment is chemotherapy. While destroying tumor cells, it would also kill normal cells, which not only causes serious toxicity and side effects to tumor patients, but also often leads to failure of chemotherapy for tumor patients.

The essence of the toxic side effects of chemotherapeutic drugs on the body is the killing of normal tissue cells by chemotherapeutic drugs. In order to effectively inhibit the non-specific killing of normal tissue cells by chemotherapeutic drugs, people use a carrier tool to encapsulate the chemotherapeutic drugs, which are released and selectively act at the tumor site. Or they can even act intracellularly within the tumor cells, thereby killing the tumor cells. Conventional microcarriers such as those synthesized with nano materials including PLGA (poly(lactic-co-glycolic acid)), liposome, PEG-PE (polyethylene glycol-phosphatidylethanolamine) have been used to encapsulate chemotherapy drugs, and proven to be able to deliver chemotherapeutic drugs to tumor sites, as well as enhance the killing effect of the drugs on tumor cells. Because the nano materials are exogenous substances relative to the body, they themselves have toxic side effects on living organisms. Meanwhile, due to their particle size specifications, the nanoparticies currently used in clinical practice can easily pass through normal tissue cells, further increasing their toxic side effects on the organisms. In addition, some nanoparticles are processed by specific materials and complex production procedures, raising the cost of nanoparticle carriers significantly. Thus they are not suitable to clinical application.

Therefore, the development of a nano-scale carrier to reduce the toxic side effects of chemotherapeutic drugs as well as inhibit the killing ability of chemotherapeutic drugs on tumor cells has become an urgent problem to be solved in the field of tumor therapy.

Chinese patent application 201110241369.8 discloses a tumor chemotherapeutic drug preparation which is prepared by encapsulating the chemotherapeutic drugs using apoptotic tumor cell vesicles. Although it has some advantages over other nano-drugs, there are still defects such as limited cell sources and high production cost.

### SUMMARY OF THE INVENTION

In order to solve the above problems, an object of the present invention is to provide a kind of erythrocyte vesicle-derived tumor therapeutic drugs, which are prepared by encapsulating tumor therapeutic drugs using a vesicle derived from erythrocyte apoptosis as a drug carrier. As targeted drugs, they can better target the tumor site, improve the drug effect, and greatly reduce the toxicity and side effects on the body of the drug delivered using an exogenous carrier. When the same chemotherapeutic drug are loaded, the one encapsulated with cell vesicles derived from apoptotic erythrocytes are more effective in treating tumors than cell vesicles derived from apoptotic tumor cells.

In present invention, a kind of tumor therapeutic drugs is provided, comprising erythrocyte vesicles and therapeutic drug component encapsulated in the erythrocyte vesicles, wherein the erythrocyte vesicle is a vesicle released by an apoptotic erythrocyte, and the therapeutic drug component are tumor therapeutic compounds as active ingredients for treating tumors.

The tumor therapeutic drug component as active ingredients for treating tumors may be any drug that is clinically effective for treating tumors, including but not limited to various chemotherapeutic agents, biological agents, certain Chinese medicine preparations, and the like. In a preferred embodiment of the present invention, it provides a tumor chemotherapeutic drug derived from the above erythrocyte vesicle, which encapsulates a chemotherapeutic component as a therapeutically active ingredient into the vesicles derived from erythrocyte apoptosis.

More specifically, the above erythrocyte vesicle-derived tumor chemotherapeutic drug provided by the present invention includes cell vesicles derived from erythrocyte apoptosis and a chemotherapeutic drug component encapsulated in the cell vesicles as an active ingredient.

According to the above erythrocyte vesicle-derived tumor chemotherapeutic drug provided by the present invention, the erythrocyte vesicle (carrier) used for encapsulating the chemotherapeutic drug component is formed by apoptosis of erythrocytes from ordinary human beings. The chemotherapeutic drug component encapsulated in the erythrocyte vesicle of the present invention may be a chemotherapeutic drug that is clinically applied before and/or after the application date of the present invention, or may be an active ingredient in a chemotherapeutic drug that is clinically applied before and/or after the application date of the present invention (may not contain or not completely contain pharmaceutical excipients). That is, in the preparation of the drug of the present invention, an active ingredient in a clinical drug composition for treating tumors may be used, or various commercially available drugs which have been clinically applied to tumor treatment may be directly used. Therefore, the dose of the drug involved in the present invention should be understood as the amount of the active ingredient of the drug. Specific chemotherapeutic drugs may be a chemotherapy agent that is clinically used to treat various types of tumor, such as: lung cancer, leukemia, ovarian cancer, colon cancer, breast cancer, bladder cancer, stomach cancer, hepatoma or glioma, and may be a single chemotherapy agent or a combination of several chemotherapy agents.

According to a preferred embodiment of the present invention, the included chemotherapeutic drug component may be in a drug form that has been used clinically, such as an injection preparation, an oral preparation or a tablet, a powder, a granule (which may be dissolved for usage; after being dissolved, it is incubated with erythrocyte vesicles so as to be encapsulated into the erythrocyte vehicles).

As a preferred embodiment of the present invention, the tumor therapeutic pharmaceutical preparation formed by encapsulating an agent in the erythrocyte vesicles has a particle size of 50-500 nm. The content of the chemotherapeutic drug component contained in the erythrocyte-derived vesicle delivered tumor chemotherapeutic drug is controlled by the amount of the chemotherapeutic drug component added to the erythrocyte culture solution during the preparation process, but the maximum drug content depends on the maximum saturation of the used chemotherapeutic drug component in the erythrocyte vesicles. Therefore, different specifications of the drug can be obtained within the range of the maximum drug content.

The present invention also provides a method of preparing the erythrocyte vesicle-derived drug, namely by encapsulating a tumor therapeutic drug in the erythrocyte vesicles by any feasible method, and the erythrocyte vesicle used is obtained by inducing apoptosis of the erythrocytes.

The erythrocyte vesicles used for encapsulating a drug can be obtained by any methods inducing apoptosis of the erythrocytes, which include but not limited to, contacting the erythrocytes with a chemotherapeutic agent, radioactive rays, or ultraviolet rays to cause the erythrocyte apoptosis. Then, a desired tumor therapeutic drug is contacted with the apoptotic erythrocytes, so as to encapsulate the drug into the apoptotic erythrocytes and obtain the pharmaceutical preparation of the present invention.

In a specific embodiment of the present invention, the method for preparing the erythrocyte-derived tumor therapeutic drug provided by the present invention comprises the steps of: administering a chemotherapeutic agent to the erythrocytes to cause their apoptosis, and collecting the drug-encapsulating vesicles released by the apoptotic erythrocytes. The drug-encapsulating vesicles are formed by encapsulating the chemotherapeutic drug for treating tumors with the erythrocyte vesicles. Or the method includes irradiating the erythrocytes with ultraviolet rays to induce erythrocyte apoptosis, collecting cell vesicles released by the apoptotic erythrocytes, and then incubating the erythrocyte vesicles with an agent for treating tumors, so that the therapeutic agent is encapsulated in the erythrocyte vehicles; and then collecting the drug-encapsulating vesicles. The drug-encapsulating vesicle is the drug formed by encapsulating the therapeutic agent with the erythrocyte vesicles. The method can also include irradiating erythrocyte with ultraviolet rays to induce their apoptosis and immediately adding a chemotherapeutic drug as an active ingredient; then collecting the drug-encapsulating vehicles released by the apoptotic erythrocytes. The drug-encapsulating vesicle is the drug formed by encapsulating the therapeutic drug with the erythrocyte vesicles.

The induction of erythrocyte apoptosis described in the present invention can be determined according to the criteria known by those skilled in the art. For example, an erythrocyte that is observed to be shrink, darken can be considered to be apoptotic. The encapsulation of a chemotherapeutic drug in the erythrocyte vesicles by incubating the erythrocyte vesicles with the chemotherapeutic drug can be achieved by allowing the erythrocyte vesicle system to which the chemotherapeutic drug is added to stand for 2-4 hours under normal temperature conditions.

In the solution of the present invention, it is preferred to use ultraviolet irradiation or a chemotherapeutic drug to induce the erythrocyte apoptosis, and collecting of the erythrocyte vesicles can be carried out using a conventional centrifuge and a high speed frozen centrifuge under low temperature conditions or at room temperature. Preferably, the cell vesicles are collected by a high speed centrifuge under low temperature conditions (about 4° C.) at a centrifugal force of 500-50,0000 g. Similarly, the collecting of the drug-encapsulating erythrocyte vesicles can also be carried out using an ultracentrifuge under low temperature conditions. Preferably, the drug-encapsulating erythrocyte vesicles are collected by a centrifuge under low temperature conditions (about 4° C.) at a centrifugal force of 500-50,0000 g. A preferred method of collecting erythrocyte vesicles is using density gradient centrifugation, which collects erythrocyte vesicles at a centrifugal force of 1000-50,0000 g. For incubation of the erythrocyte vesicles obtained by ultraviolet irradiation of the erythrocytes and a chemotherapeutic drug for treating tumors, the chemotherapeutic drug is preferably added to the erythrocyte vesicles in an amount close to the maximum saturation of the chemotherapeutic drug in the erythrocyte vesicle carrier. They are incubated at normal temperature, so that the chemotherapeutic drug is finally encapsulated by the erythrocyte vesicles.

For the collected drug-encapsulating erythrocyte vesicles, a pharmaceutical preparation such as an injection preparation, can be prepared according to conventional methods. For example, the collected drug-encapsulating erythrocyte vesicles are suspended in physiological saline to prepare an injection.

The drug-encapsulating erythrocyte-derived vesicles provided by the present invention can be administered according to conventional clinical treatment methods. For example, the drug-encapsulating erythrocyte-derived vesicle preparation can be targeted to bladder tumor by transurethral bladder perfusion for bladder cancer. For ovarian tumor, it can be administered directly by intraperitoneal injection. The administrated dose can be applied at or below the conventional dose of the administered chemotherapeutic drug component.

According to the drug of the present invention, different specifications (content of the active ingredient) of pharmaceutical preparation can be obtained by controlling the amount of the chemotherapeutic drug component added, thereby facilitating the administration to tumor patients at different stages. The content of the chemotherapeutic drug component in the obtained drug vesicles can be determined according to actual practical situations such as the nature of the drug selected, the administration of the drug to a specific tumor, and the prevalence stage of the tumor patient to be treated.

According to the type of tumor to be treated and the chemotherapeutic drug used as an active ingredient and according to the above-described method for preparing a erythrocyte-derived tumor chemotherapeutic drug, a person skilled in the art can select an appropriate method for inducing erythrocyte apoptosis, a method for collecting drug-encapsulating erythrocyte vehicles and the suitable conditions such as ratio of the amount of erythrocyte vesicles to the amount of chemotherapeutic drug for treating tumors, as long as the content of the chemotherapeutic drug as the active ingredient encapsulated in the finally obtained erythrocyte vesicles is sufficient to exert the desired therapeutic effect.

According to another aspect of the present invention, it is also provided a pharmaceutical composition comprising the tumor therapeutic drug of the present invention. The pharmaceutical composition can be prepared by adding various pharmaceutically and/or physiologically acceptable excipients and/or additives to the drug of the present invention. Preferred pharmaceutical compositions are provided in the than of a liquid formulation containing PBS buffer or physiological saline.

As basic knowledge of this research field, the spherical structure of cells is maintained by the centripetal pulling force formed by the protein fibrils of cytoskeleton. When the cell is subject to the stimulus of external conditions (for example: chemotherapeutic drugs or ultraviolet radiation), cell apoptosis can occur. At the same time, part of the protein fibrils of the cytoskeleton attached to the cell membrane are broken and lose adhesion, and the centripetal pulling force suddenly disappears. Then part of the cell membrane structure expands outward and protrudes under the effect of the outgoing pulling force, and encapsulates the cellular content, which is released to the extracellular substructure between the cell and molecules in the form of cell vesicles. Recently, the cell vesicles encapsulating a chemotherapeutic drug have been prepared into a pharmaceutical preparation, which is named “drug-encapsulating vesicles”. The cell vesicles used are mostly the tumor cell-derived cell vesicles, having a particle size of 100-1000 nm, whereas the erythrocyte-derived cell vesicles provided by the present invention have a particle size of 50-500 nm. Compared with tumor cell-derived drug-encapsulating vesicles, the erythrocyte-derived drug-encapsulating vesicles of the present invention have a good uniformity and can more effectively pass through tumor capillary tissue and kill tumor cells (at the tumor site of the body, where the gaps between the cells that make up its blood vessels increase to 100-780 nm), but will not cause any damage to normal tissues (the permeability of normal body tissue is usually 5-10 nm). Under the premise of encapsulating the same chemotherapeutic drug and the same amount of the drug, the erythrocyte-derived cell vesicles have a larger quantity and smaller particle sizes than the tumor cell-derived vehicles, and can be easily absorbed by the tumor cells. They have better therapeutic effect than the tumor cell-derived vehicles, and overcomes some major defects of tumor cell-derived vesicles: First of all, compared with tumor cells, the number of erythrocytes in the body is much larger, and it is not necessary to perform large-scale expansion of cultivation since erythrocytes can be directly obtained from the body, which greatly reduces the production cost; secondly, a tumor cell-derived drug-encapsulating vesicle can only treat the cancer related to the same tumor cells, whereas the erythrocyte-derived drug-encapsulating vesicles can simultaneously treat a variety of cancers, thereby expanding the type and scope of the tumor treatment. Therefore, for the erythrocyte-derived tumor chemotherapeutic drug preparation of the present invention, the administered dosage of the pharmaceutical preparation for treating tumors may be appropriately reduced if needed in practical use. Or to achieve the same therapeutic effect, a smaller content of the chemotherapeutic drug component may be selected for encapsulation according to the actual situation during tumor treatment, compared with the dosage administered when the chemotherapy drug component is applied directly.

More specifically, the erythrocyte vesicle-derived tumor therapeutic drug disclosed by the present invention has the following advantages:

1. By encapsulating the tumor therapeutic drug with the erythrocyte vesicles, the drug of the present invention can reduce the toxicity and side effects of the administered exogenous carrier on the body, and the drug can be targeted to the tumor site, which alleviates the toxicity and side effects of the drug on the body and improves the drug efficacy.

2. According to the erythrocyte-derived tumor chemotherapeutic drug of the present invention, the drug-encapsulating erythrocyte vehicles have a smaller and more uniform particle size (50-500 nm) than the drug-encapsulating tumor cell vesicles (100-1000 nm). Therefore, the drug-encapsulating erythrocyte vesicles are more stable and easier to enter the tumor cells than the drug-encapsulating tumor cell vesicles. In the case of equal drug content, the drug-encapsulating erythrocyte vesicles have larger quantities and smaller particle sizes than the drug-encapsulating tumor cell vesicles. Therefore, the drug-encapsulating erythrocyte vesicles are more easily absorbed by the tumor cells, and the therapeutic effect is better than that of the drug-encapsulating tumor cell vesicles.

3. For the same number of erythrocytes and tumor cells, the cell vesicle yield produced by erythrocytes is higher than that produced by tumor cells.

4. The structure of the drug-encapsulating erythrocyte vesicles of the present invention is more stable than that of the drug-encapsulating tumor cell-derived vesicles. Because, after treatment with 1% TrtionX-100 and different concentrations of SDS solution, the number of erythrocyte-derived vesicles and the killing effect on tumor cells are superior to those of tumor cell-derived vesicles. Meanwhile, storage at 4° C. can prolong the activity of drug-encapsulating erythrocyte-derived vesicles, and the neutral or weakly alkaline (pH 7˜pH 8.5) environment is more conducive to the storage of drug-encapsulating erythrocyte-derived vesicles.

5. Compared with tumor cells, erythrocytes are derived from human blood, and can be directly taken from human blood. No large-scale cell culture is required, and the cost is low and the operation is simple.

6. Compared with tumor cells, erythrocyte vesicles can target most tumors, while the tumor cell vesicles only target the tumor that produces the tumor cell vesicles, which expands the type and scope of tumors to be treated, reduces costs and risks, and simplify the operation.

### DESCRIPTION OF THE DRAWINGS

FIG. 1-1 is a field emission scanning electron microscopy of the drug-encapsulating erythrocyte-derived vehicles, showing the morphology of erythrocyte-derived vehicles;

FIG. 1-2 is a field emission scanning electron microscopy of the drug-encapsulating H22 tumor cell vehicles, showing the morphology of H22 tumor cell-derived vesicles;

FIG. 1-3 shows the detection results of particle size of erythrocyte-derived vesicle, showing the particle sizes of erythrocyte-derived vesicles;

FIG. 1-4 shows the detection results of H22-MPs, showing the particle sizes of H22 cell-derived vesicles;

FIG. 1-5 shows the yields of drug-encapsulating erythrocyte vesicles and drug-encapsulating H22 tumor cell vesicles in a unit cell;

FIG. 2 shows the contents of methotrexate encapsulated in erythrocyte vesicles, and H22 tumor cell vesicles;

FIG. 3 shows the killing effect of drug-encapsulating erythrocyte vesicles on various tumor cells;

FIG. 4-1 shows the difference in number between the drug-encapsulating erythrocyte vesicles and the drug-encapsulating H22 tumor cell vesicles after storage for 24 hours in different organic solvents;

FIG. 4-2 shows the killing effect of the drug-encapsulating erythrocyte vesicles and the drug-encapsulating H22 tumor cell vesicles on H22 cells after storage for 24 hours in different organic solvents;

FIG. 5-1 shows the number of the drug-encapsulating erythrocyte vesicles under different environmental conditions as a function of storage days;

FIG. 5-2 shows the number of the drug-encapsulating erythrocyte vesicles under different pH conditions as a function of storage days;

FIG. 6-1 shows the killing effect of drug-encapsulating erythrocyte vesicles on H22 cells under different environmental conditions after different storage days;

FIG. 6-2 shows the killing effect of drug-encapsulating erythrocyte vesicles on H22 cells under different pH conditions after different storage days;

FIG. 7 shows the survival curve of the BABL/c mouse ascites hepatoma model;

FIG. 8 shows the survival curve of the C57BL/6 mouse lung cancer model;

FIG. 9-1 shows the glutamic-pyruvic transaminase and serum creatinine content of BABL/c mice in each experimental group;

FIG. 9-2 shows the APTT and PT values in the coagulation of BABL/c mice in each experimental group;

FIG. 10 shows the killing function of methotrexate-encapsulating vesicles obtained by stimulating erythrocytes with calcium ions;

FIG. 11 shows the killing function of the erythrocyte drug carrier obtained by treating the erythrocytes with a hypertonic solution.

### DETAILED DESCRIPTION OF THE INVENTION

In order to illustrate the technical solution of the present invention, and confirm that the erythrocyte-derived cell vesicles can encapsulate a chemotherapeutic drug, and the chemotherapeutic drug-encapsulating erythrocyte vehicles can effectively kill the tumor cells without producing obvious toxic and side effects in the body, the invention will be further described in conjunction with the drawings and examples. The following examples are not intended to limit the invention in any way.

The “tumor cell vesicle” used in the present invention is produced by an apoptotic tumor cell, without encapsulating a chemotherapeutic drug, “erythrocyte vesicle” is produced by an apoptotic erythrocyte, without encapsulating a chemotherapeutic drug; a cell vesicle encapsulating a drug is described as a drug-encapsulating tumor cell vesicle or a drug-encapsulating erythrocyte vesicle.

Various tumor cells, drugs, laboratory animals, equipments and partial solution preparation used in the examples are listed as follows:

Cells: H22 mouse hepatoma cells (H22 cells), A2780 human ovarian cancer cells, human breast tumor cell line MCF-7, human lung cancer cell line A549, human gastric cancer cell line MGC-803, human colorectal cancer T84, human hepatoma cell line HepG2, human ovarian cancer cell line Ho-8910, human cervical cancer cell line Hela, human prostate cell line PC-3, human esophageal cancer cell line EC109, human nasopharyngeal cancer cell line CNE, human kidney cancer cell line A498, human squamous carcinoma cell line A-431, human lymphoblastoid cells NCI-BL2009, human fibrocarcinoma cells HT-1080, human bladder cancer cell line T24, Human leukemia cell line K562, human promyelocytic leukemia cells HL60, human melanoma cells A875, human acute lymphocytic leukemia cells MOLT-4, human erythroleukemia HEL and human glioma cell line U251. All of them can be purchased from the US ATCC company or China Center for Type Culture Collection (CCTCC).

Reagents and drugs: 3μm commercial standard magnetic beads (magnetic beads for cell vesicle relative counting) were purchased from SIGMA-ALDRICH company of USA, and methotrexate (MTX) was purchased from Tongji Hospital affiliated to Tongji Medical College (Wuhan).

Others not specifically stated were all commercially available.

Experimental animals; BALM mice, C57BL/6 mice were purchased from Hubei Medical Laboratory Animal Research Center under the Hubei Provincial Center for Disease Control and Prevention;

Equipments: Sirion 200 field emission scanning electron microscope, Nano ZS90 nanometer particle size potentiometer, BDFACSCanto II flow cytometer, UltiMate 3000 high performance liquid chromatography.

Preparation of Solutions

Cell vesicle rupture solution: 20 mM Tirs-HCl, 1% Triton X-100, 150 mM NaCl, 1 mM EDTA pH 7.5.

Note: In the following examples, the control group is abbreviated as control, the MTX-encapsulating erythrocyte vesicle administration group is abbreviated as Ery-MPs (MTX), the MTX-encapsulating H22 tumor cell vesicle administration group is abbreviated as H22-MPs (MTX), the MTX encapsulating Lewis lung cancer cell vesicle administration group is abbreviated as LLC-MPs (MTX), and the chemotherapy drug methotrexate administration group is abbreviated as MTX.

### [Example 1]: Morphological Observation and Particle Size Measurement of the Chemotherapeutic Drug-Encapsulating Cell Vesicles Obtained by Incubating the Cell Vesicles Produced by UV-Induced Apoptosis of Mouse Hepatoma Cells and Mouse Erythrocytes with a Chemotherapeutic Drug

1. Experimental Materials and Reagents

H22 mouse hepatoma cells, normal mouse erythrocytes, methotrexate

2. Experimental Steps

1) H22 mouse hepatoma cells were cultured in 1640 cell culture medium solution until the total number of the cells reach 3×10⁸; a certain amount of normal mouse blood was collected in an anticoagulant tube, and the same amount of PBS was added and mixed, centrifuged at 2000 rpm for 5 minutes and the supernatant was removed. The erythrocytes were washed 3 times with Ca²⁺, Mg²⁺-free Hank's solution, centrifuged for 5 minutes at 2000 rpm for the first 2 times, and centrifuged for 10 minutes at 2000 rpm for the last time. Then the supernatant was removed. The erythrocyte concentration was calculated using a hemocytometer, and a total of 3×10⁸ mouse erythrocytes were taken out therefrom.

Three parts of the above H22 cells and normal mouse erythrocytes were prepared respectively. Each part of the above cells was transferred to 10 ml of 1640 cell culture medium, and then irradiated with ultraviolet rays for 1 h. Then the chemotherapeutic drug methotrexate was added to make the drug concentration in the culture solution reach 1 mg/ml, and then incubated for 20 h.

2) The above obtained H22-MPs (MTX) and Ery-MPs (MTX) culture solutions were subjected to stepwise centrifugations to obtain the supernatant respectively, that is, centrifuged at 2000 rpm and 5000 rpm for 10 minutes respectively, to remove cells and debris.

The above obtained two kinds of supernatants were centrifuged at a centrifugal force of 14000 g for 1.5 minutes, and the supernatants after centrifugation were further centrifuged at a centrifugal force of 14000 g for 1 h. The supernatants were discarded, and the surfaces of the precipitates were rinsed with PBS, and finally resuspended in 1 ml of PBS, respectively, to obtain three parts of H22-MPs (MTX) and 3 parts of Ery-MPs (MTX).

3) One part of H22-MPs (MTX) or Ery-MPs (MTX) was treated before scanning and photoing by field emission scanning electron microscopy: cell vesicle fixation, dehydration, freeze vacuum drying, and conductive treatment before scanning by electron microscopy.

Fixation of cell vesicles: The surfaces of H22-MPs (MTX) and Ery-MPs (MTX) were rinsed with PBS, and then centrifuged at a centrifugal force of 14000 g for 1 h to obtain H22-MPs (MTX) and Ery-MPs (MTX) precipitates, respectively. They were resuspended with 500 μl of 2.5% glutaraldehyde, and protected from light overnight at 4° C., and the fixative (2.5% glutaraldehyde) was absorbed and removed. Then the vesicles were washed with 1 ml of PBS for 10 min, and centrifuged at a centrifugal force of 14000 g for 1 h, which was repeated once. Then, they were fixed with 500 μl of 2.5% glutaraldehyde for another 1 h, and washed with 1 ml of PBS for 10 min, and then centrifuged at a centrifugal force of 14,000 g for 1 h, which was repeated once.

Dehydration of cell vesicles: The above H22-MPs (MTX) and Ery-MPs (MTX) precipitates obtained by immobilization with 2.5% glutaraldehyde were dehydrated with acetone and isoamyl acetate mixture solution (acetone: isoamyl acetate solution=1:1) for 10 min, respectively, then centrifuged at a centrifugal force of 14000 g for 1 h. The resulting two precipitates were dehydrated with isoamyl acetate for 30 min, respectively, and then centrifuged at a centrifugal force of 14000 g for 1 h.

Freeze vacuum drying of cell vesicles: The H22-MPs (MTX) and Ery-MPs (MTX) after dehydration treatment were resuspended in a small amount of sterile water, immediately placed in a refrigerator to be pre-cooled at 4° C. for 10 min, then subjected to freezing treatment in a refrigerator at −20° C. for 2 h. Thereafter, the frozen samples were subjected to a freeze vacuum drying treatment.

Conductive treatment of cell vesicles: A suitable copper conductive table was selected and aluminum conductive adhesive was pasted on the table, and carbon conductive adhesive was pasted on the scanning surface. Then, the dried samples were uniformly applied to the surface of the aluminum conductive paste, respectively. Then the sample surfaces were prayed with platinum.

After the above treatment steps, the samples were sent to an observation table of the field emission scanning electron microscope to perform a scanning pretreatment. Then, the samples were scanned by field emission and photo-recorded from an appropriate field of view.

4) Another part of H22-MPs (MTX), and Ery-MPs (MTX) solutions were centrifuged for another 1 h at a centrifugal force of 14000 g, respectively. The resulting precipitates were resuspended in 1 ml of PBS, respectively. Then, the particle sizes of the samples were measured by a nanoparticle size potential analyzer (DLS).

5) The remaining part of H22-MPs (MTX) and Ery-MPs (MTX) solutions were mixed with a certain volume of 3 μm commercial standard magnetic bead solution with known concentration to prepare a flow counting solution, and then counted by a flow cytometry. The concentrations of H22-MPs (MTX) and Ery-MPs (MTX) solutions can be calculated according to the ratio of the number of cell vesicles obtained by flow counting to the number of 3 μm magnetic beads with the known concentration. Finally, the yields of H22-MPs (MTX) and Ery-MPs (MTX) were calculated, respectively.

3. Experimental Results

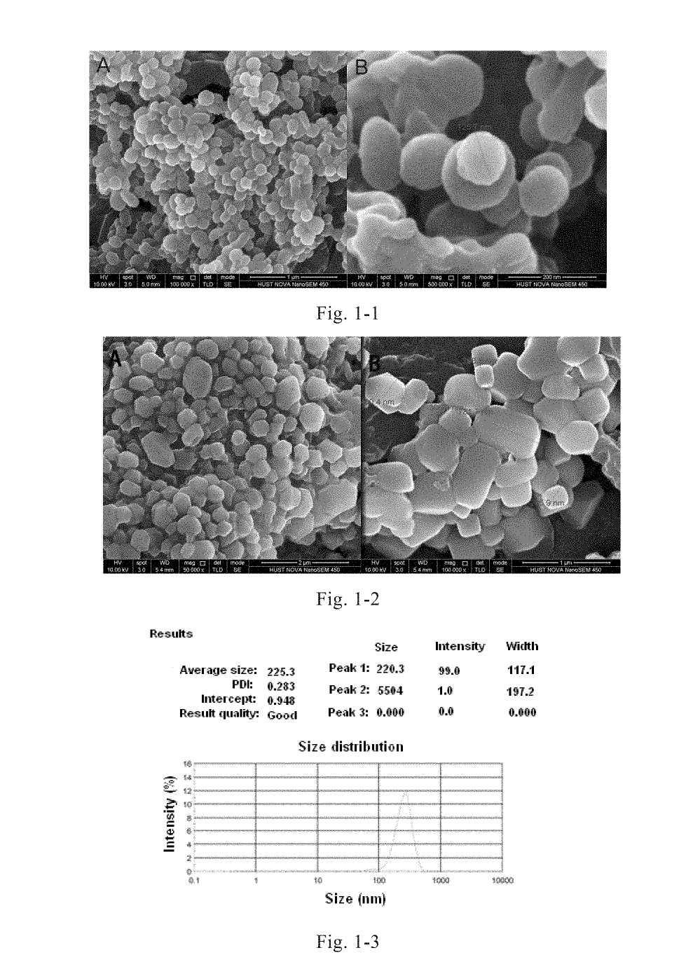

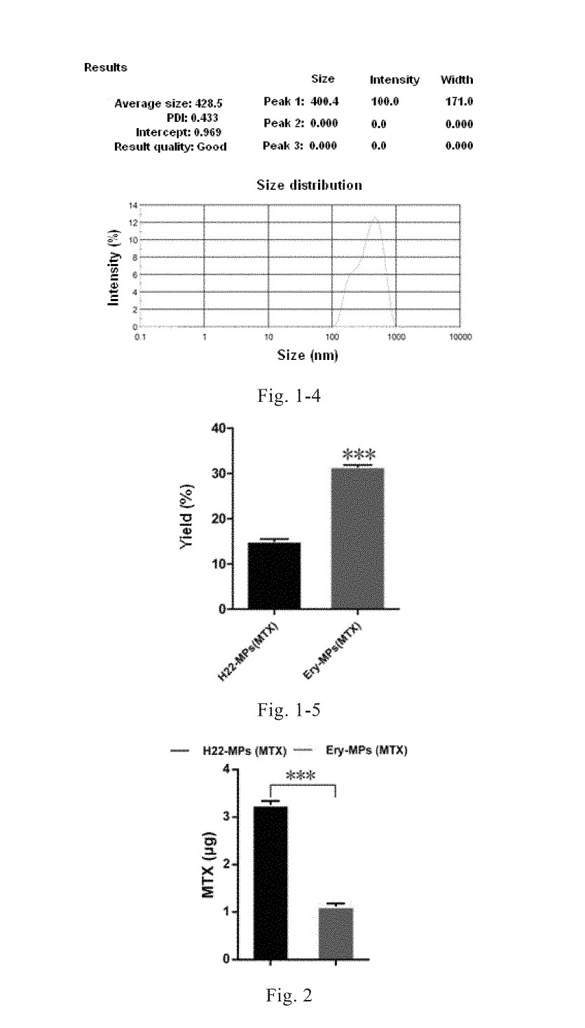

The results of FIG. 1-1 and FIG. 1-2 show that after the sample treatment for scanning electron microscopy, the surface morphology of H22-MPs (MTX) changed to some extent, whereas Ery-MPs (MTX) could basically maintain ellipsoidal shape. This indicates indirectly that Ery-MPs (MTX) is easier to maintain ellipsoidal structure than H22-MPs (MTX). Meanwhile, the particle size of Ery-MPs (MTX) was relatively uniform, whereas the particle size of H22-MPs (MTX) was relatively dispersed. The results of particle size measurement in FIG. 1-3 and FIG. 1-4 show that the average particle size of Ery-MPs (MTX) was about 225 nm, and the average particle size of H22-MPs (MTX) was about 428 nm. The average particle size of H22-MPs (MTX) is about twice the average particle size of Ery-MPs (MIX).

The results in FIG. 1.5 show that the yield of the cell vesicles produced in 1×10⁸ H22 cells was about 14.67%; and the yield of the cell vesicles produced in 1×10⁸ erythrocytes was about 31.45%. The yield of the erythrocyte-derived cell vesicles is approximately 2.15 times as much as that of the 1122 cell-derived cell vesicles.

### [Example 2]: Detection of Content of Chemotherapeutic Drug (Methotrexate) Encapsulated in H22 Cell-Derived Vesicles and Mouse Erythrocyte-Derived Vesicles

1. Experimental Materials and Reagents

H22 mouse hepatoma cells, mouse erythrocytes, methotrexate, high performance liquid chromatography.

2. Experimental Steps

1) H22 mouse hepatoma cells were cultured with 1640 cell culture medium to make the total amount of cells reach 1×10⁸; mouse blood was collected according to the method of Example 1, erythrocytes were isolated, and the erythrocyte concentration was calculated. A total of 1×10⁸ mouse erythrocytes were taken therefrom.

2) H22-MPs (MTX) and Ery-MPs (MTX) were prepared, isolated and extracted according to the method of Example 1, respectively.

3) H22-MPs (MTX) and Ery-MPs (MTX) were counted according to the method of Example 1, respectively, and the concentrations of H22-MPs (MTX) solution and Ery-MPs (MTX) solution were calculated, respectively.

4) Each of 1×10⁷ H22-MPs (MTX) and Ery-MPs (MTX) were taken and diluted with PBS to 1 ml, then centrifuged at a centrifugal force of 14000 g for 1 h. The supernatants were removed to obtain precipitates, which were washed and resuspended with 500 μL of PBS, and repeated twice.

The above-obtained 500 μL of H22-MPs (MTX) and Ery-MPs (MIX) solutions were centrifuged at a centrifugal force of 14000 g for 1 h, and the supernatants were removed to obtain precipitates, which were lysed with a cell vesicle rupture solution, incubated on ice for 30 minutes, disrupted with a sonicator for at least 40 seconds, and then centrifuged at 1000 g for 5 minutes, to obtain the supernatants.

Two volumes of acetonitrile was added to the supernatants and violently vibrated to precipitate the proteins, followed by centrifugation at 1000 g for 3 minutes, to obtain the supernatant. Then, four volumes of chloroform was added to the supernatant to remove the lipid substances, followed by centrifugation at 2000 g for 10 minutes.

The above two supernatants were collected, and the contents of the drug in the supernatants were determined by high performance liquid chromatography, respectively, wherein, the mobile phase was 0.05 M KH₂PO₄, 10% acetonitrile, pH 6.6, and the flow rate was 1 ml/min. The column temperature was 40° C. and the UV absorption wavelength of methotrexate was 304 nm. Finally, the contents of methotrexate in the two collected supernatants were calculated, respectively.

3. Experimental Results

The results of FIG. 2 show that the content of methotrexate encapsulated in 1×10⁷ Ery-MPs (MTX) was about 1.12 μg, and the content of methotrexate encapsulated in 1×10⁷H22-MPs (MTX) was about 3.43 m. The content of methotrexate encapsulated in H22 cell-derived vesicles is 3.0625 times as many as that in same amount of erythrocyte-derived vesicles.

### [Example 3]: Killing Effect of Methotrexate Encapsulated in the Vesicles Produced by Human Erythrocyte Apoptosis on Various Tumor Cells

1. Experimental Materials and Reagents

Different human tumor cell lines include: A2780 human ovarian cancer cells, human breast tumor cell line MCF-7, human lung cancer cell line A549, human gastric cancer cell line MGC-803, human colorectal cancer T84, human hepatoma cell line HepG2, human ovarian cancer cell line Ho-8910, human cervical cancer cell line Hela, human prostate cell line PC-3, human esophageal cancer cell line EC109, human nasopharyngeal cancer cell line CNE, human kidney cancer cell line A498, human squamous carcinoma cell line A-431, human lymphoblastoid cells NCI-BL2009, human fibrocarcinoma cells HT-1080, human bladder cancer cell line T24, Human leukemia cell line K562, human promyelocytic leukemia cells HL60, human melanoma cells A875, human acute lymphocytic leukemia cells MOL7-4, human erythroleukemia HEL and human glioma cell line U251.

Normal human erythrocytes, chemotherapeutic drug methotrexate.

2. Experimental Steps

1) The above tumor cell lines were cultured in DMEM serum culture medium, respectively, for tumor cell killing experiments. Human blood was collected according to the erythrocyte collection method of Example 1, the corresponding erythrocytes were collected, and the erythrocyte concentrations of the erythrocyte packed cell volume were calculated. The preparation method of Ery-MPs (MTX) was the same as in Example 1.

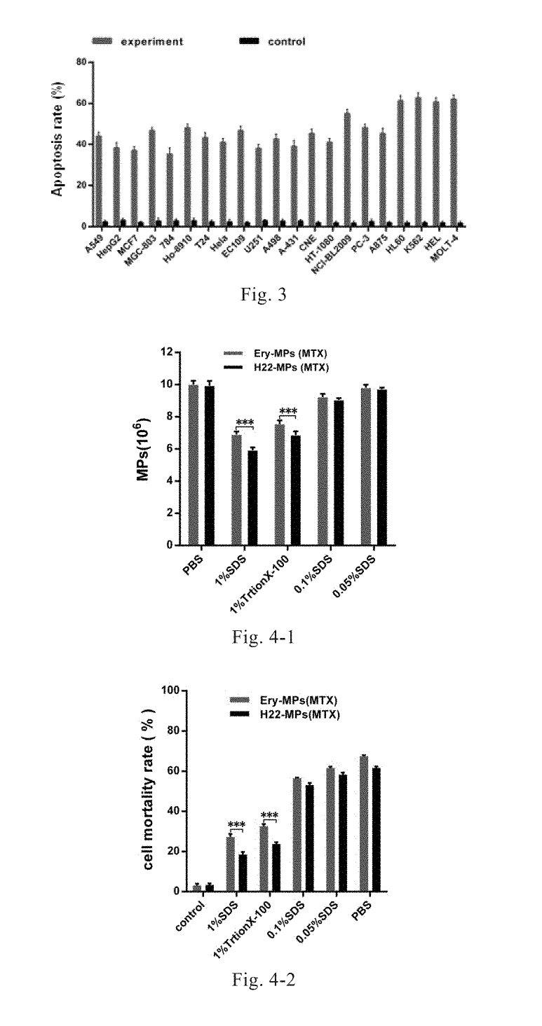

2) 1×10⁵ tumor cells were normally cultured in the control group, while Ery-MPs (MTX) was added in the culture system in the experimental groups, (in the experimental group, 2×10⁶ Ery-MPs (MTX) was added to 1×10⁵ of tumor cells). After incubation of Ery-MPs (MTX) and the tumor cells for 48 hours, the liquid in each well was transferred to an EP tube, centrifuged at 500 g for 6 minutes, and the cell pellet was collected. The supernatant was discarded and the cell pellet was resuspended and washed with PBS for once. Then, the supernatant was discarded and the cell pellet was resuspended by adding 100 μl of Bind Buffer. 1.5 μl of Annexin V and 1 μl of PI dye were added in the dark, and mixed well. After incubating for 15 minutes at room temperature, 200 μl of Bind Buffer was added. Then, the death state of the tumor cells of the experimental group and the control group was detected by flow cytometry, and the results are shown in FIG. 3.

3. Experimental Results

It can be seen from FIG. 3 that the methotrexate-encapsulating human erythrocyte vesicles had a good killing effect on various tumor cells, indicating that the erythrocyte-derived vesicles encapsulating tumor chemotherapeutic drug provided by the present invention is applicable for various tumor cells, and has good effect.

### [Example 4]: Comparison of Stability and Effect on Tumor Cell Killing Between Ery-MPs (MTX) and H22-MPs (MTX) Stored in Different Organic Solvents

1. Experimental Materials and Reagents

H22 mouse hepatoma cells, mouse erythrocytes, methotrexate, 1% TrtionX-100, 1% SDS, 0.1% SDS, 0.01% SDS, PBS.

Ultraviolet device belongs to a conventional cell clean bench.

2. Experimental Steps

1) H22 mouse hepatoma cells were cultured with 1640 cell culture medium to make the total amount of cells reach 4×10⁹; mouse blood was collected according to the method of Example 1, erythrocytes were isolated, and the erythrocyte concentration was calculated. A total of 4×10⁹ mouse erythrocytes were taken therefrom.

2) H22-MPs (MTX) and Ery-MPs (MTX) were prepared, isolated and extracted according to the method of Example 1, respectively. Then H22-MPs (MTX) and Ery-MPs (MTX) were resuspended in 2 ml of PBS respectively and counted according to the method of Example 1. The concentrations of H22-MPs (MTX) and Ery-MPs (MTX) solutions were calculated, respectively.

3) 15 samples of H22-MPs (MTX) and Ery-MPs (MTX) solutions were prepared respectively, ensuring that the total number of the cell vesicles in each solution was 1×10⁷. Then each solution was diluted to 1 ml, centrifuged at a centrifugal force of 14000 g for 1 h. The supernatant was removed to obtain a packed cell precipitate solution. 24 ml of 1% TrtionX-100, 1% SDS, 0.1% SDS, 0.01% SDS solution were prepared respectively, each solution was divided into six equal parts, and put into the corresponding EP tube.

4) Three equal parts of 1% TrtionX-100, 1% SDS, 0.1% SDS, 0.01% SDS solution were added to 12 samples of above H22-MPs (MTX) solution and Ery-MPs (MTX) packed cell precipitate solutions respectively. The remaining 3 samples of H22-MPs (MTX) solution and Ery-MPs (MTX) packed cell precipitate solution were added with 1 ml of PBS, respectively. Each of the solutions was mixed well, and then stored at 4° C. for 24 hours and centrifuged at a centrifugal force of 14000 g for 1 h. The supernatants were removed, and the packed cell precipitate solutions were obtained and resuspended in 1 ml of PBS, respectively.

5) The concentrations of H22 cell-derived vesicles and mouse erythrocyte-derived vesicles in the corresponding EP tube were detected using a flow cytometry, and converted to the numbers of H22-MPs (MTX) and Ery-MPs (MTX) in the corresponding EP tube.

6) 15 samples of H22-MPs (MTX) solution and Ery-MPs (MTX) solution were prepared respectively, ensuring that the total number of H22-MPs (MTX) in each sample was 1×10⁷, and the total number of Ery-MPs (MTX) in each sample was 3×10⁷ (According to the detection results of Example 2, it was ensured that the drug content of the erythrocyte-derived vesicles was the same as the drug content of the H22 cell-derived vesicles), and then each solution was diluted to 1 ml, centrifuged at a centrifugal force of 14000 g for 1 h. The supernatant was removed and the packed cell precipitate solution was obtained. And it was treated according to the procedure of 4), and finally each packed cell precipitate solution was resuspended in 100 μL of 1640 medium and mixed well.

7) Three equal samples of 10 μL H22-MPs (MTX), Ery-MPs (MTX) medium resuspension solutions were taken from each of 1% TritionX-100 group, 1% SDS group, 0.1% SDS group, 0.01% SDS, PBS group respectively, and then co-cultured with 5×10⁴ H22 tumor cells in a 48-well plate (ensuring that the final culture volume was 1 mL). For the control group corresponding to the experimental groups, 5×10⁴ H22 tumor cells were added to the corresponding wells (ensuring that the final culture volume was 1 mL), cultured for 36 hours. Then the liquid in each well was transferred to an EP tube, centrifuged at 500 g for 6 minutes, and the cell pellet was collected. The supernatant was discarded and the cells was resuspended and washed once with PBS. Then the supernatant was discarded again and the cell pellet was resuspended by adding 100 μl of Bind Buffer. 1.5 μl of Annexin. V and 1 μl of PT dye were added in the dark and mixed well. After incubating for 15 minutes at room temperature, 200 μl of Bind Buffer was added. Then, the death state of the tumor cells in the experimental groups and the control group was detected by flow cytometry.

3. Experimental Results

The results in FIG. 4-1 show that the effects of 1% SDS, 1% TrtionX-100, 0.1% SDS, and 0.05% SDS on cell vesicles decreased in the order. Under the same conditions, the number of erythrocyte-derived vesicles in mice was significantly higher than H22 cell-derived vesicles.

The results of FIG. 4-2 show that the killing effects of the drug-encapsulating vesicles on the tumor cells in the 0.05% SDS, 0.1% SDS, 1% Trition X-100, and 1% SDS groups enhanced in the order, and the killing effect of Ery-MPs (MTX) on the tumor cells was significantly stronger than that of H22-MPs (MTX). The above experiments demonstrate that mouse erythrocyte-derived vesicles are significantly more stable than H22 cell-derived vesicles.

### [Example 5]: Comparison of Effects of Different Environmental Conditions and Storage Times on the Stability of Drug-Encapsulating Erythrocyte Vesicles

1. Experimental Materials and Reagents

Mouse erythrocytes, methotrexate, 1 mM NaOH, 1 mM HCl.

Ultraviolet device belongs to a conventional cell clean bench.

2. Experimental Steps

1) According to the method of Example 1, mouse blood was collected and the erythrocytes were isolated which concentration was calculated. A total of 1.5×10⁹ mouse erythrocytes were taken therefrom.

2) Ery-MPs (MTX) was prepared, isolated and extracted according to the method of Example 1. The above obtained Ery-MPs (MTX) was centrifuged at a centrifugal force of 14000 g for 1 h. The supernatant was removed to obtain a packed cell precipitate solution, which was then divided into two equal parts.

3) One part of Ery-MPs (MIX) packed cell solution was divided into 12 equal parts. Each equal part was resuspended in 1 ml of PBS and placed in an EP tube. Four groups of conditions were tested, including storage at 37° C., storage at 4° C., strong light treatment, mild vibration treatment. Each group had three replicates.

4) Another part of mouse Ery-MPs (MTX) packed cell solution was divided into 15 equal parts. Each part was resuspended in 500 μL of PBS and placed in an EP tube. Among them, 12 parts of above 500 μL Ery-MPs (MTX) suspension were prepared into solutions with pH of 5.5, 7, 8.5, and 10 (making up the volume to 3 ml), respectively. Each group had three replicates. Another three parts of 500 μL of Ery-MPs (MTX) suspension were added with PBS to make up the volume to 3 ml.

5) The storage time gradient was provided, including storage for 1 day, 2 days, 3 days, 4 days, 5 days, 6 days and 7 days, respectively. The corresponding Ery-MPs (MTX) concentration of each sample stored at each time gradient was measured by flow cytometry, which was converted to the total amount of Ery-MPs (MTX) in the corresponding EP tube.

3. Experimental Results

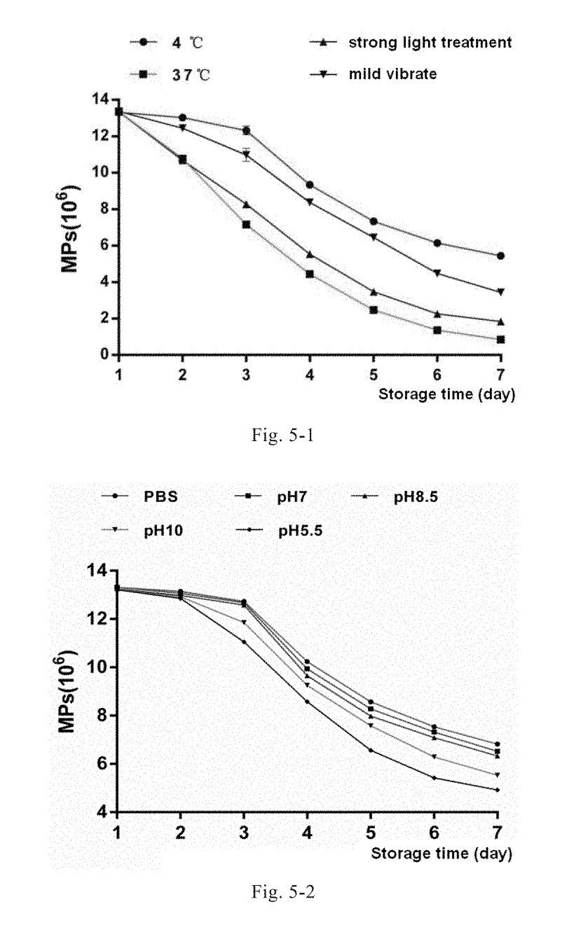

The results in FIG. 5-1 show that storage at 4° C. and mild vibration had less effect on the quantities of Ery-MPs (MTX), and the total amount of Ery-MPs (MTX) decreased slowly during the first 3 days. From the 4th day on, the number of Ery-MPs (MTX) decreased sharply.

The results of FIG. 5-2 show that five groups tested under different environmental conditions have basically the same trend on the quantity change of Ery-MPs (MTX). Among them, the total amounts of Ery-MPs (MTX) in PBS, pH 7 and pH 8.5 groups decreased slowly during the first 3 days, and 3 days later, the numbers of Ery-MPs (MTX) of the three groups decreased sharply. There was no significant difference in the quantities of Ery-MPs (MTX) among the three groups. The total amount of Ery-MPs (MTX) decreased slowly in the first 2 days in pH 10 and pH 5.5 groups, and there was no significant difference in the total amount of Ery-MPs (MTX) in PBS, pH 7 and pH 8.5 groups. From the 3rd day on, the number of Ery-MPs (MTX) in pH 10 and 5.5 groups decreased sharply, and the decreasing trend was more obvious than that in PBS, pH 7 and pH 8.5 groups. Among them, the number of Ery-MPs (MTX) in pH 5.5 group decreased more significantly than that in pH 10 group.

### [Example 6]: Killing Effect of Ery-MPs (MTX) on Tumor Cells Under Different Environmental Conditions and Storage Times

1. Experimental Materials and Reagents

Mouse erythrocytes, methotrexate, 1 mM NaOH, 1 mM HCl.

Ultraviolet device belongs to a conventional cell clean bench.

2. Experimental Steps

1) According to the method of Example 1, mouse blood was collected and the erythrocytes were isolated whose concentration was calculated. A total of 1.5×10⁹ mouse erythrocytes were taken therefrom.

2) Ery-MPs (MTX) was prepared, isolated and extracted according to the method of Example 1. The above obtained Ery-MPs (MTX) was centrifuged at a centrifugal force of 14000 g for 1 h. The supernatant was removed to obtain a packed cell precipitate solution, which was then divided into two equal parts.

3) One part of Ery-MPs (MTX) packed cell solution was divided into 12 equal parts. Each equal part was resuspended in 1 ml of PBS and placed in an EP tube. Four groups of conditions were provided, including storage at 37° C., storage at 4° C., strong light treatment, mild shock treatment. Each group had three replicates.

Another part of Ery-MPs (MTX) packed cell solution was divided into 15 equal parts. Each part was resuspended in 500 μL of PBS and placed in an EP tube. Among them, 12 parts of the 500 μL Ery-MPs (MTX) suspension were prepared into solutions with pH of 5.5, 7, 8.5, and 10 (making up the volume to 3 ml), respectively. Each group had three replicates. Another three parts of the 500 μL of Ery-MPs (MTX) suspension were added with PBS to make up the volume to 3 ml.

4) According to the above experimental steps, four parts of Ery-MPs (MTX) were treated under the above conditions.

5) The storage time gradient was provided, including storage for 0 day, 1 day, 2 days, 3 days, respectively. Three equal parts of Ery-MPs (MTX) in each group were co-cultured with H22 tumor cells (2×10⁶ Ery-MPs (MTX) was added to 1×10⁵ H22 tumor cells, ensuring that the final culture medium volume was 1 mL). In the control group, the corresponding number of H22 tumor cells was cultured (ensuring that the final culture volume was 1 mL) for 36 hours. The liquid in each well was transferred to an EP tube, centrifuged at 500 g for 6 minutes, and the cell pellet was collected. The supernatant was discarded and the cells were resuspended and washed once with PBS. Then the supernatant was discarded again and the cell pellet was resuspended in 100 μl of Bind Buffer. 1.5 μl of Annexin V and 1 μl of PI dye were added in the dark and mixed well. After incubating for 15 minutes at room temperature, 200 μl of Bind Buffer was added. The death state of the tumor cells in the experimental groups and the control group was detected by flow cytometry.

3. Experimental Results

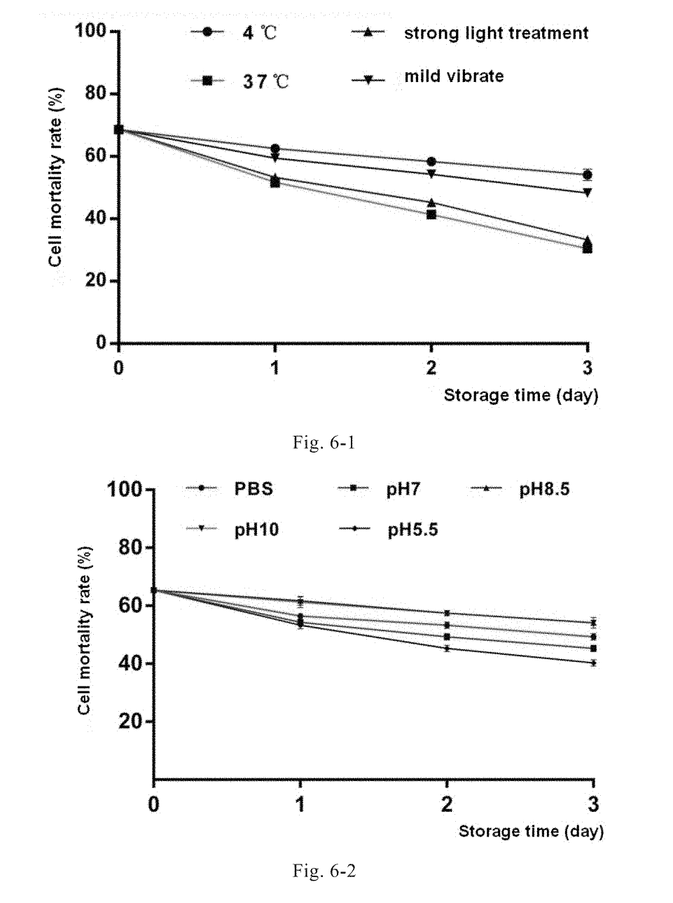

The results of FIG. 6-1 show that storage at 4° C. was more suitable to the preservation of Ery-MPs (MTX). The activity of Ery-MPs (MTX) on killing tumor cells decreased after storage for 3 days, but it did not decrease significantly.

The results of FIG. 6-2 show that Ery-MPs (MTX) was more stable in neutral or weak alkaline (pH7-pH8.5) storage environment. The activity of Ery-MPs (MTX) on killing tumor cells decreased after storage for 3 days, but it did not decrease significantly.

The above experiments show that the drug-encapsulating erythrocyte vesicles could be stored for about 3 days at 4° C., and will be more stable in neutral or weak alkaline (pH 7-pH 8.5) environment.

### [Example 7]: Comparison of Effect of Ery-MPs (MTX) and H22-MPs (MTX) on Tumor Growth Inhibition and Prolongation of Survival Time of Hepatoma Ascites Model Mice

1. Experimental Materials and Reagents

H22 mouse hepatoma cells, mouse red blood cells, chemotherapy drug methotrexate, and 40 BALM mice were used.

2. Experimental Steps

1) H22 cells were cultured to a total of 2×10⁸ cells, and the culture method was the same as in Example 1. The blood of the mice was collected, the erythrocytes were isolated according to the erythrocyte isolation method of Example 1, and the number of cells was counted until the total number of the cells reached 3×10⁸.

2) 2×10⁷ H22 hepatoma cells and 3×10⁷ mouse erythrocytes were taken from the above cells. According to the preparation, isolation and extraction method of drug-encapsulating cell vesicles of Example 1, the corresponding H22-MPs (MTX) and Ery-MPs (MTX) were prepared, isolated and extracted, and resuspended in 100 μL physiological saline, respectively. Then, the numbers of H22-MPs (MTX) and Ery-MPs (MTX) were calculated by the cell vesicle counting method of Example 1.

2×10⁶ H22-MPs (MTX) and 6×10⁶ Ery-MPs (MTX) were taken respectively (according to the measurement results of Example 2, ensuring that the drug content of Ery-MPs (MTX) was equal to the drug content of H22-MPs (MTX)), resuspended with physiological saline to an volume of 200 μL which was determined as the daily dose of the drug for one mouse.

3) On the first day, 5×10⁴ H22 cells were intraperitoneally inoculated into BALB/c mice, and a total of 40 BALB/c mice were injected to obtain the one with H22 cell hepatoma ascites model, which were randomly divided into four groups equally, namely experimental group 1, experimental group 2, experimental group 3 and control group. The BALB/c mice in experimental group 1 were intraperitoneally injected with H22-MPs (MTX); the BALM mice in experimental group 2 were intraperitoneally injected with Ery-MPs (MTX); the BALB/c mice in experimental group 3 were intraperitoneally injected with methotrexate at a dose of 1 μg/g; and the BALB/c mice in the control group were intraperitoneally injected with 200 μL of 0.9% (g/ml) physiological saline.

4) From the 4th day, according to the same injection procedure, the experimental groups were injected with H22-MPs (MTX), Ery-MPs (MTX) and chemotherapy drug (methotrexate) once a day for 7 consecutive days, respectively, while the control group was injected with 200 μL 0.9% (g/ml) physiological saline once a day for 7 consecutive days. From the 11th day, all of the BALB/c mice were fed normally, and the survival status of the BALB/c mice in each group was observed.

3. Experimental Results

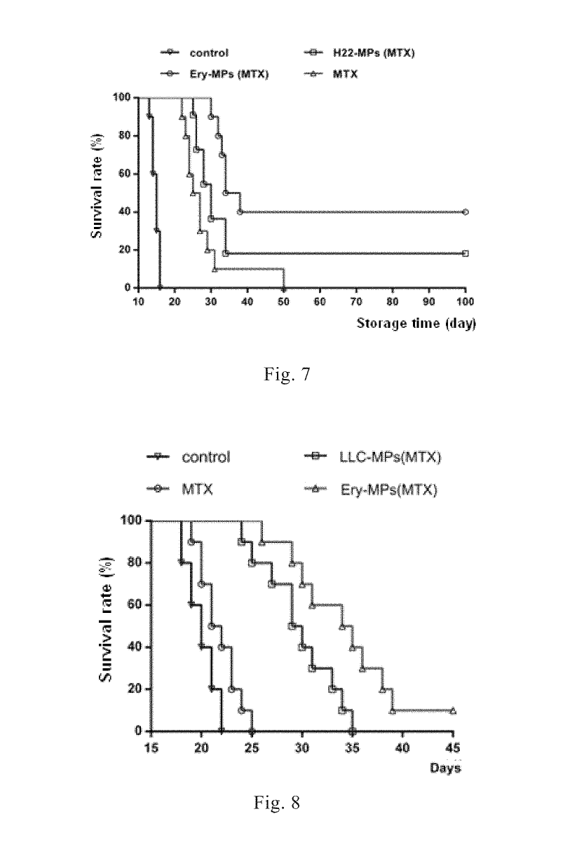

The results of FIG. 7 show that death of the mice in the control group occurred from the 13th day and all of them died on the 16th day. In the experimental group 1, BALB/c mice injected intraperitoneally with H22-MPs (MTX) died from the 25th day, and a total of 8 mice died until the 34th day. The survival rate was 20%. In the experimental group 2, BALB/c mice injected intraperitoneally with Ery-MPs (MTX) died from the 27th day, and a total of 6 mice died until the 33th day. The survival rate was 40%. In the experimental group 3, BALB/c mice injected intraperitoneally with methotrexate died from the 24th day, and a total of 9 mice died until the 31th day. The remaining one died on the 50th day.

The above results demonstrate that the therapeutic effect of methotrexate-encapsulating erythrocyte vesicles is better than that of methotrexate-encapsulating tumor cell vesicles, and much better than that of single methotrexate treatment group.

### [Example 8]: Comparison of Inhibition of Solid Lung Cancer Growth in Mice and Prolongation of Survival Time of the Lung Cancer Model Mice Between Ery-MPs (MTX) and Methotrexate-Encapsulating Lewis Lung Cancer Cell Vesicles (LLC-MPs (MTX))

1. Experimental Materials and Reagents

Mouse Lewis lung cancer cells, normal mouse erythrocytes, methotrexate, 32 C57BL/6 mice.

2. Experimental Steps

1) Lewis cells were cultured to a total of 2×10⁸ cells, and the culture method was the same as in Example 1. The blood of the mice was collected, the erythrocytes were isolated according to the erythrocyte isolation method of Example 1. The number of cells was counted until the total number of the cells reached 3×10⁸.

2) 2×10⁷ Lewis lung cancer cells and 3×10⁷ mouse erythrocytes were taken from the above cells. According to the preparation, isolation and extraction method of drug-encapsulating cell vesicles of Example 1, the corresponding LLC-MPs (MTX), Ery-MPs (MTX) were prepared, isolated and extracted, and resuspended in 100 μL physiological saline, respectively. Then the numbers of LLC-MPs (MTX) and Ery-MPs (MTX) were calculated by the cell vesicle counting method of Example 1.

2×10⁶ LLC-MPs (MTX) and 6×10⁶ Ery-MPs (MIX) were taken respectively, (according to the measurement results of Example 2, ensuring that the drug content of Ery-MPs (MIX) was equal to the drug content of LLC-MPs (MIX)), resuspended with physiological saline to an volume of 200 μL, which was determined as the daily dose of the drug for one mouse.

3) On the 1st day, 1×10⁶ Lewis cells were inoculated into a C57BL/6 mouse via tail vein, and a total of 32 C57BL/6 mice were injected to obtain the C57BL/6 mice of Lewis cell lung cancer model which were randomly divided into four groups equally, namely, experiment group 1, experiment group 2, experiment group 3 and control group. Among them, the C57BL/6 mice in the experimental group 1 were injected with LLC-MPs (MTX) via tail vein; the C57BL/6 mice in the experimental group 2 were injected with Ery-MPs (MIX) via tail vein; the C57BL16 mice in the experimental group 3 were injected with methotrexate at a dose of 0.5 μg/g; the C57BL/6 mice in the control group were injected via tail vein with 200 μL of 0.9% (g/ml) physiological saline.

4) From the 5th day, according to the same injection procedure, the experimental groups were injected with LLC-MPs (MTX), Ery-MPs (MTX) and chemotherapy drug (methotrexate) once a day for 10 consecutive days respectively, while the control group was injected with 200 μL 0.9% (g/ml) physiological saline once a day for 10 consecutive days. From the 15th day, all of the C57BL/6 mice were fed normally, and the survival status of the C57BL/6 mice in each group was observed.

3. Experimental Results

The results of FIG. 8 show that Ery-MPs (MTX) has a significantly better therapeutic effect on lung cancer in mice than LLC-MPs (MIX). The therapeutic effect of methotrexate on lung cancer in mice is much poorer than that of Ery-MPs (MTX) and LLC-MPs (MTX).

### [Example 9]: Toxicity and Side Effect Test of Red Blood Cell Vesicles Encapsulating Chemotherapeutic Drug on the Body

1. Experimental Materials and Reagents

The H22 mouse hepatoma cells and mouse erythrocytes used were the same as in Example 1, the chemotherapy drug methotrexate, and 32 BALB/c mice.

2. Experimental Steps

1) H22 cells were cultured to a total of 2×10⁸ cells, and the culture method was the same as in Example 1. The blood of the mice was collected, the erythrocytes were isolated according to the erythrocyte isolation method of Example 1, and the number of cells was counted until the total number of the cells reached 3×10⁸.

2) 2×10⁷ H22 hepatoma cells and 3×10⁷ mouse erythrocytes were taken from the above cells. According to the preparation, isolation and extraction methods of cell vesicles of Example 1, the corresponding H22-MPs (MIX), Ery-MPs (MTX) were prepared, isolated and extracted, and resuspended in 100 μL physiological saline, respectively. Then the numbers of H22-MPs (MTX) and Ery-MPs (MTX) were calculated by the cell vesicle counting method of Example 1.

2×10⁶ H22-MPs (MTX) and 6×10⁶ Ery-MPs (MTX) were taken respectively, (according to the measurement results of Example 2, ensuring that the drug content of Ery-MPs (MTX) was equal to the drug content of H22-MPs (MTX)), resuspended with physiological saline to an volume of 200 which was determined as the daily dose of the drug for one mouse.

3) The prepared H22-MPs (MTX) was injected into 8 BALB/c mice via tail vein respectively, as the experimental group 1; the prepared Ery-MPs (MTX) was injected into 8 BALM mice via tail vein respectively, as the experimental group 2; 8 mice BALB/c mice were injected with methotrexate via tail vein at a dose of 1 μg/g, as the experimental group 3; 8 mice BALB/c mice were injected with 0.9% (g/ml) physiological saline via tail vein, as the control group. According to the same injection procedure, the experimental groups were injected with H22-MPs (MTX), Ery-MPs (MTX) and methotrexate respectively once a day for 7 consecutive days, while the control group was injected with 200 μL 0.9% (g/ml) physiological saline once a day for 7 consecutive days.

4) On the 8th day, venous blood was taken from the mice in the experimental group 1, the experimental group 2, the experimental group 3 and the control group, and the content of glutamic-pyruvic transaminase, creatinine in serum and the content of APTT and PT in the blood were detected. The body weights of the mice were weighed.

3. Experimental Results

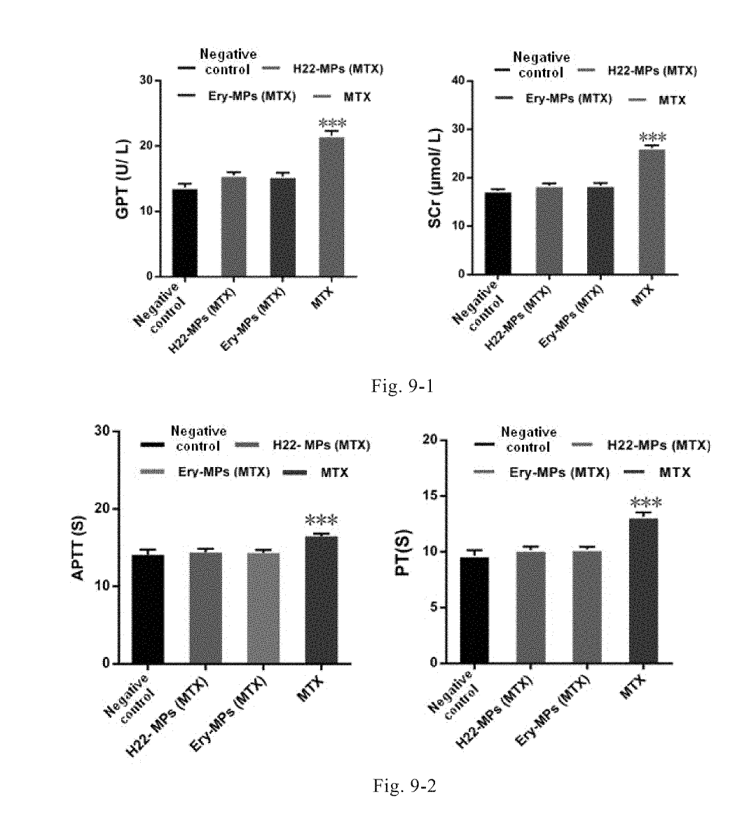

The results of FIG. 9-1 and FIG. 9-2 show that average values of glutamic-pyruvic transaminase (GPT) in BABL/c mice of the negative control injected with physiological saline, and the administration groups of Ery-MPs (MTX), H22-MPs (MTX) and methotrexate were 13.63 U/L, 14.50 U/L, 14.32 U/L, and 21.57 In, respectively. The average values of serum creatinine content of the four groups of BABL/c mice were 17.29 μmol/L, 17.78 μmol/L, 17.34 μmol/L, and 26.13 μmol/L, respectively. The average values of activated partial prothrombin time (APTT) in BABL/c mice of the four groups were 14.13 s, 14.4 s, 14.33 s, and 16.5 s, respectively. The average values of plastin time (PT) in BABL/c mice of the four groups were 9.67 s, 10.13 s, 10.20 s, and 13.13 s, respectively.

It is known from the above data that compared with the negative control injected with physiological saline, there is no significant difference in the glutamic-pyruvic transaminase and serum creatinine content and the APTT and PT values in blood coagulation in BABL/c mice of Ery-MPs (MTX) and H22-MPs (MTX) administration groups, and they are all within the ranges of normal values. In relative to the above three groups, the contents of glutamic-pyruvic transaminase and serum creatinine and the values of APTT a) and PT in blood coagulation are significantly higher in the BABL/c mice of the methotrexate administration group.

In summary, compared with the chemotherapeutic drug component, the methotrexate-encapsulating erythrocyte vesicles significantly reduced toxicity and side effects on the body, as well as the methotrexate-encapsulating tumor cell vesicles.

### [Example 10]: Comparison of Killing Functions of the Drug Encapsulated in the Vesicles Released by Ultraviolet-Induced Erythrocyte Apoptosis and the Vesicles Released by Calcium Ion-Stimulated Erythrocyte Apoptosis

1. Experimental Materials and Reagents

H22 mouse hepatoma cells and mouse erythrocytes used were the same as those in Example 1, chemotherapeutic drug methotrexate, and the Annexin V-FITC/PI cell apoptosis detection kit.

Ultraviolet device belongs to a conventional cell clean bench.

2. Experimental Steps

1) The blood of the mice was collected, and the erythrocytes were isolated according to the erythrocyte isolation method of Example 1, and the number of the cells was calculated.

2) 3×10⁷ mouse erythrocytes were taken from the above cells, and divided into the following three groups:

In the first group, the corresponding Ery-MPs (MTX) were prepared, isolated and extracted according to the preparation, isolation and extraction method of the cell vesicles of Example 1, and resuspended in physiological saline.

In the second group, the mouse cells were adjusted with HBSS to a final concentration of 1.5×10⁶/ml, added with CaCl₂ to a final concentration of 390 mM, incubated at 37° C. for 30 min, centrifuged at 2000 rpm for 10 min to remove CaCl₂, resuspended in 1640 medium, and added with methotrexate to a final concentration of 1 mg/ml and incubated for another 5 h. According to the preparation, isolation and extraction method of Example 1, the methotrexate-encapsulating erythrocyte vesicles that were released by calcium ion-stimulated erythrocytes were obtained by preparation, isolation and extraction procedures.

In the third group, the mouse cells were adjusted with HBSS to a final concentration of 1.5×10⁶/ml, added with CaCl₂ to a final concentration of 390 mM, incubated at 37° C. for 30 min, centrifuged at 2000 rpm for 10 min to remove CaCl₂, resuspended in 1640 medium, subjected to UV irradiation for 1 h, and then added with methotrexate to a final concentration of 1 mg/ml, and incubated for another 5 h. According to the preparation, isolation and extraction method of Example 1, the methotrexate-encapsulating erythrocyte vesicles that were released by co-stimulation of erythrocyte with calcium ions and ultraviolet rays were obtained by preparation, isolation and extraction procedures.

3) The above three groups of methotrexate-encapsulating erythrocyte vesicles were counted using the cell vesicle counting method of Example 1.

4) The killing effect of the three groups of methotrexate-encapsulating vesicles released by erythrocytes on H22 cells was detected by the method of Example 3.

3. Experimental Results

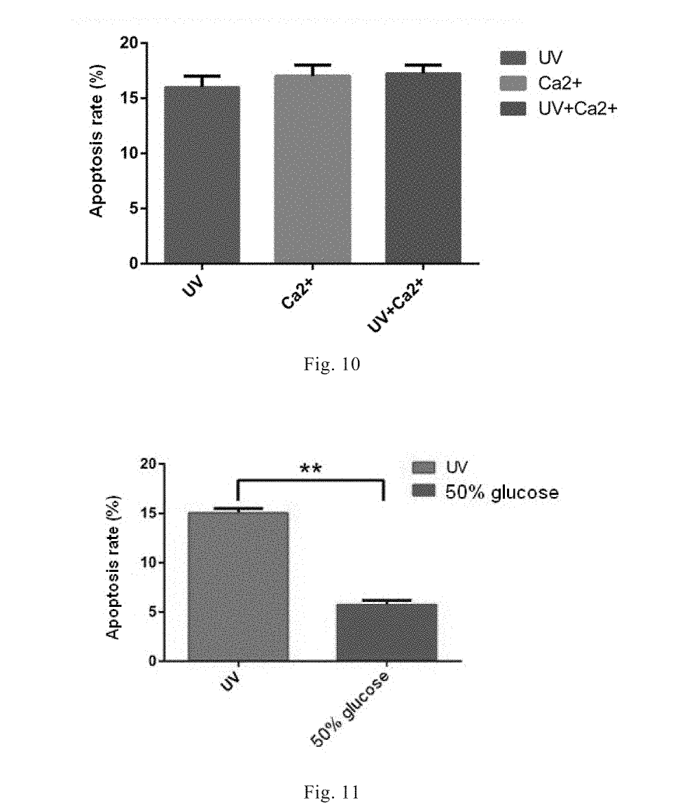

As shown in FIG. 10, the killing effect of the drug delivery system obtained by Ca²⁺ stimulation of mouse erythrocytes was comparable to the killing effect of the methotrexate-encapsulating vesicles obtained by UV-induced apoptosis, while the killing effect of methotrexate-encapsulating vesicles obtained by both Ca′ stimulation and UV treatment of mouse erythrocytes was not improved significantly. This demonstrates that there is no difference in the killing effect between the drug-encapsulating vehicles obtained by UV-induced erythrocyte apoptosis and calcium ion stimulation.

### [Example 11]: Comparison of Killing Functions Between Chemotherapeutic Drug-Encapsulating Erythrocyte Vesicles and Other Erythrocyte-Derived Drug-Encapsulating Carriers

1. Experimental Materials and Reagents

H22 mouse hepatoma cells and mouse erythrocytes used were the same as those in Example 1, chemotherapeutic drug methotrexate, and the Annexin V-FITC/PI cell apoptosis detection kit.

Ultraviolet device belongs to a conventional cell clean bench.

2. Experimental Steps

1) The blood of the mice was collected, and the erythrocytes were isolated according to the erythrocyte isolation method of Example 1, and the number of the cells was calculated.

2) 3×10⁷ mouse erythrocytes were taken from the above cells, and divided into the following two groups:

According to the preparation, isolation and extraction method of the cell vesicles of Example 1, the corresponding Ery-MPs (MTX) were prepared, isolated, extracted, and resuspended in physiological saline.

The mouse cells were mixed well with 50% glucose injection to the final concentration of 1.5×10⁶/ml, and allowed to stand at room temperature for 40 min, centrifuged at 2000 rpm for 10 min, and the supernatant was discarded to obtain dehydrated erythrocytes. The corresponding volume of MTX injection (1 mg/ml) prepared by 1640 medium was added, vibrated well, allowed to stand for 30 min, and centrifuged at 500 rpm for 10 min. The supernatant was discarded and the volume of the erythrocytes was made up by adding physiological saline and mixing well, to obtain the methotrexate-encapsulating erythrocyte carrier (RBCs-MTX solution).

3) The drug contents of the above two groups of erythrocyte-derived drug carriers were calculated using the method of Example 2, respectively.

4) According to the method of Example 3, the killing effects of methotrexate encapsulated in the two groups of erythrocyte-derived carrier on H22 cells were detected, ensuring that the drug loading of the carrier (MTX-RBCS) obtained by treating erythrocytes with 50% glucose injection in each well was identical to the drug loading of 2×10⁶ erythrocyte vesicles.

3. Experimental Results

As shown in FIG. 11, the killing effect of the drug carrier system obtained by treating the mouse erythrocytes with 50% glucose injection was not so good as the killing effect of the methotrexate-encapsulating vesicles obtained by ultraviolet-induced apoptosis. This demonstrates that the killing function of the drug-encapsulating erythrocyte vesicles is stronger than that of erythrocyte-derived drug carrier system obtained by high osmotic pressure treatment of erythrocytes.

Summary of Other Experimental Results:

When the erythrocyte vesicles produced by the apoptosis of normal mouse erythrocytes obtained in Example 1 are used to encapsulate other chemotherapeutic drugs, such as cyclophosphamide, 5-fluorouracil, gemcitabine, doxorubicin, pirarubicin, paclitaxel, hydroxycamptothecin, vincristine, ancitabine, carboplatin and cisplatin, the results are consistent with the above examples. Similarly, when the erythrocyte vesicles produced by the apoptosis of ordinary human erythrocytes obtained in Example 3 are used to encapsulate other chemotherapeutic drugs, such as cyclophosphamide, 5-fluorouracil, gemcitabine, doxorubicin, pirarubicin, paclitaxel, hydroxycamptothecin, vincristine, ancitabine, carboplatin and cisplatin, the obtained results are also consistent with the above examples.

For the erythrocytes isolated from the blood of mice with hepatoma and ascites, the erythrocyte vesicles are obtained according to the preparation method of Example 1 and used to encapsulate a chemotherapeutic drug, such as methotrexate, cyclophosphamide, 5-fluorouracil, gemcitabine, doxorubicin, pirarubicin, paclitaxel, hydroxycamptothecin, vincristine, ancitabine, carboplatin and cisplatin, the obtained results are consistent with the above examples. Similarly, when the erythrocyte vesicles produced by apoptosis of erythrocytes from the corresponding tumor patients in. Example 3 are used to encapsulate a chemotherapeutic drug, such as methotrexate, cyclophosphamide, 5-fluorouracil, gemcitabine, doxorubicin, pirarubicin, paclitaxel, hydroxycamptothecin, vincristine, ancitabine, carboplatin and cisplatin, the obtained results are consistent with the above examples.

1. A tumor therapeutic drug comprising: erythrocyte vesicles and a therapeutic drug component encapsulated in the erythrocyte vesicles; wherein the erythrocyte vesicles are released by apoptotic erythrocytes; and the therapeutic drug component is an active ingredient for treating tumors.

2. The tumor therapeutic drug of claim 1, wherein the erythrocyte vesicles are obtained by contacting human-derived erythrocytes with any one selected from the group consisting of chemotherapeutic agents, radiation rays, and/or ultraviolet rays to induce erythrocyte apoptosis and collecting the vesicles released.

3. The tumor therapeutic drug of claim 1, wherein the tumor therapeutic drug formed by encapsulating the therapeutic drug component with the erythrocyte vesicles has a particle size of 50 to 500 nm.

4. The tumor therapeutic drug of claim 1, wherein the tumor therapeutic drug component is a chemotherapeutic drug.

5. The tumor therapeutic drug of claim 4, wherein the chemotherapeutic drug is one or more selected from the group consisting of chemotherapeutic drugs for treating lung cancer, colon cancer, ovarian cancer, leukemia, gastric cancer, liver cancer, breast cancer, bladder cancer, and glioma tumor.

6. The tumor therapeutic drug of claim 4, wherein the chemotherapeutic drug is selected from the group consisting of methotrexate, cyclophosphamide, 5-fluorouracil, gemcitabine, doxorubicin, pirarubicin, paclitaxel, hydroxycamptothecin, vincristine, ancitabine, carboplatin and cisplatin.

7. The tumor therapeutic drug of claim 6, wherein the chemotherapeutic drug is methotrexate.

8. The tumor therapeutic drug of claim 1, wherein the drug is prepared by the following method:
administering the chemotherapeutic drug component to the erythrocytes to cause their apoptosis, and collecting the drug-encapsulating vehicles released by the apoptotic erythrocytes, to obtain the tumor therapeutic drug; or
irradiating the erythrocytes with ultraviolet rays to induce the erythrocyte apoptosis, collecting the erythrocyte vesicles released by the apoptotic erythrocytes, and incubating the erythrocyte vesicles with the tumor therapeutic drug component as an active ingredient for treating tumors, so that the tumor therapeutic drug component are encapsulated by the erythrocyte vehicles, to obtain the tumor therapeutic drug; or
irradiating the erythrocytes with ultraviolet rays and immediately adding a chemotherapeutic drug as an active ingredient to induce the erythrocyte apoptosis, and collecting the drug-encapsulating vehicles released by the apoptotic erythrocytes, to obtain the tumor therapeutic drug.

9. The tumor therapeutic drug of claim 8, wherein the preparation method further comprises the step of storing the obtained tumor therapeutic drug at 4° C., pH 7.0-8.5.

10. A pharmaceutical composition, comprising the tumor therapeutic drug of claim 1 and pharmaceutically or physiologically acceptable excipients and/or additives.

11. The pharmaceutical composition of claim 10, wherein the pharmaceutical composition is a liquid preparation containing PBS buffer or physiological saline.
